(19) United States
(12) Patent Application Publication
(10) Pub. No.: US 2008/0299538 A1
(43) Pub. Date: Dec. 4, 2008

(54) Pathogen Inactivation of Whole Blood

(75) Inventors: Raymond P. GOODRICH, Lakewood, CO (US); Dennis J. HLAVINKA, Arvada, CO (US); Heather L. REDDY, Denver, CO (US)

Correspondence Address: CaridianBCT, Inc.;Mail Stop: 810 1F2, 10811 WEST COLLINS AVE, LAKEWOOD, CO, 80215, US

(73) Assignee: CARIDIANBCT BIOTECHNOLOGIES, LLC, Lakewood, CO (US)

(21) Appl. No.: 12/182,280

(22) Filed: Jul. 30, 2008

Related U.S. Application Data
(63) Continuation-in-part of application No. 10/377,524, filed on Feb. 28, 2003.
(60) Provisional application No. 60/953,374, filed on Aug. 1, 2007.

(51) Int. Cl. A01N 1/02; A61J 1/10

(52) U.S. Cl. 435/2; 604/410

(57) ABSTRACT

This invention is directed toward a method of pathogen inactivating whole blood. The steps include collecting whole blood from a donor into a bag; illuminating the whole blood with light at a sufficient energy so that an alloxazine photosensitizer present in the whole blood may be photolyzed to inactivate any pathogens which may be present in the whole blood; and storing the pathogen inactivated whole blood. The invention also includes a method of separating the pathogen inactivated whole blood into components.

### CROSS REFERENCE TO RELATED APPLICATIONS

This application claims benefit under 35 U.S.C. 119(e) of U.S. Provisional Application No. 60/953,374, filed Aug. 1, 2007 and claims benefit under 35 U.S.C 120 of U.S. application Ser. No. 10/377,524, filed Feb. 28, 2003.

### BACKGROUND

Contamination of blood supplies with infectious microorganisms such as HIV, hepatitis and other viruses and bacteria presents a serious health hazard for those who must receive transfusions of whole blood or administration of various blood components such as platelets, red cells, plasma, Factor VIII, plasminogen, fibronectin, anti-thrombin III, cryoprecipitate, human plasma protein fraction, albumin, immune serum globulin, prothrombin complex, plasma growth hormones, and other components isolated from blood. Blood screening procedures may miss contaminants, and sterilization procedures which do not damage cellular blood components but effectively inactivate all infectious viruses and other microorganisms have only recently been developed.

Photosensitizers, or compounds which absorb light of a defined wavelength and transfer the absorbed energy to an electron acceptor may be a solution to the above problems. Photosensitizers may be used to inactivate infectious microorganisms or other undesirable elements such as white blood cells which may be contaminating a blood product, without damaging the desirable components of blood.

There are many photosensitizer compounds known in the art to be useful for inactivating undesirable elements. Examples of such photosensitizers include porphyrins, psoralens, dyes such as neutral red, methylene blue, acridine, toluidines, Ravine (acriflavine hydrochloride) and phenothiazine derivatives, coumarins, quinolones, quinones, anthroquinones and endogenous photosensitizers such as riboflavin.

Whole blood collected from volunteer donors for transfusion recipients is typically separated into platelets, plasma and red blood cells using various known methods. If a photosensitizer is used to inactivate pathogens in blood, whole blood is usually separated into its components before each component is subjected to a pathogen inactivation procedure. This is because the red blood cell component of whole blood absorbs a large portion of the light needed to activate certain photosensitizers, increasing the chance of any pathogens which may be present not getting inactivated. To deliver light to the whole blood in the amount necessary to inactivate pathogens in the presence of red blood cells would be high enough to cause damage to the other components in the whole blood. It is to this problem of pathogen reducing whole blood before it is separated into components that the present invention is directed.

### BRIEF SUMMARY OF THE INVENTION

This invention is directed toward a method of pathogen inactivating whole blood. The steps include collecting whole blood from a donor into a bag; illuminating the whole blood with light at a sufficient energy so that an alloxazine photosensitizer present in the whole blood may be photolyzed to inactivate any pathogens which may be present in the whole blood; and storing the pathogen inactivated whole blood. The invention also includes a method of separating the pathogen inactivated whole blood into components.

### BRIEF DESCRIPTION OF THE DRAWINGS

FIG. 1 is a schematic view of a set of bags and a flow diagram of a process of the present invention.

FIG. 2 is a schematic view of another set of bags and another process of the present invention.

FIG. 3 is a schematic view of a blood component expresser which may be used with the present invention.

FIG. 4 is a cross-sectional view of a whole blood separation apparatus which may be used with the present invention.

FIG. 5 is a schematic view and partial cross-section of a set of separation and collection bags designed for cooperating with the automated whole blood separation apparatus of FIG. 4.

FIG. 6 is a flow diagram of a process for separating pathogen inactivated whole blood into components using the apparatus of FIG. 4.

FIG. 7 is a schematic view of another set of separation and collection bags designed for cooperating with another automated whole blood separation apparatus.

FIG. 8 is a cross-sectional view of another whole blood separation apparatus which may be used with the present invention.

FIG. 9 is a top view of the rotor of the separation apparatus of FIG. 8.

FIG. 10 is a flow diagram of a process for separating pathogen inactivated whole blood into components using the apparatus of FIG. 8.

FIGS. 11A and 11B are graphs of the log reduction of both enveloped and non-enveloped viruses as a function of illumination energy.

FIG. 12 is a graph of hemolysis during refrigerated storage of treated red blood cells as a function of illumination energy.

FIG. 13 is a graph of ATP levels during refrigerated storage of treated red blood cells as a function of illumination energy.

FIG. 14 is a graph of mean osmotic fragility of treated red blood cells during refrigerated storage as a function of illumination energy.

FIG. 15 is a graph of potassium levels in treated and untreated whole blood over 5 days of storage as whole blood at room temperature as a function of illumination energy.

FIGS. 16A and 16B are graphs of plasma quality during frozen storage as a function of illumination energy.

FIG. 17 is a table of measures of platelet quality for both treated and untreated platelets as a function of illumination energy.

### DETAILED DESCRIPTION

A “photosensitizer” useful in this invention is defined as any compound which absorbs radiation at one or more defined wavelengths and subsequently utilizes the absorbed energy to carry out a chemical process.

Endogenous photosensitizers may be used in this invention. The term “endogenous” means naturally found in a human or mammalian body, either as a result of synthesis by the body or because of ingestion as an essential foodstuff (e.g. vitamins) or formation of metabolites and/or byproducts in vivo. When endogenous photosensitizers are used, particularly when such photosensitizers are not inherently toxic or do not yield toxic photoproducts after photoradiation, no removal or purification step is required after decontamination, and the decontaminated product can be directly administered to a patient.

Examples of such endogenous photosensitizers which may be used in this invention are alloxazines such as 7,8-dimethyl-10-ribityl isoalloxazine (riboflavin), 7,8,10-trimethylisoalloxazine (lumiflavin), 7,8-dimethylalloxazine (lumichrome), isoalloxazine-adenine dinucleotide (flavin adenine dinucleotide [FAD]) and alloxazine mononucleotide (also known as flavin mononucleotide [FMN] and riboflavin-5-phosphate). The term “alloxazine” includes isoalloxazines.

Use of endogenous isoalloxazines as photosensitizers to inactivate blood and blood components are described in U.S. Pat. Nos. 6,258,577 and 6,277,337 both issued to Goodrich et al., and herein incorporated by reference to the amount not inconsistent.

The amount of photosensitizer to be mixed with the whole blood to be inactivated will be an amount sufficient to adequately inactivate any pathogen-associated nucleic acids which may be present in the fluid, but less than a toxic (to the blood components) or insoluble amount. A pathogen may be defined as any undesirable element found in blood, such as bacteria, virus and white blood cells.

If riboflavin is used as the photosensitizer, it may be added to the whole blood at a final concentration of between about 50-500 μM. Pathogen-associated nucleic acid includes any undesirable nucleic acid such as nucleic acid contained in white blood cells, bacteria or viruses. Nucleic acids include either deoxyribonucleic acid (DNA), ribonucleic acid (RNA) or both.

The whole blood to which the photosensitizer has been added is exposed to light of the appropriate wavelength to activate the photosensitizer and to substantially inactivate and cause permanent damage to the pathogen-associated nucleic acids. Substantially permanent damage means that the nucleic acids will not undergo self-repair or replication during storage or upon infusion into a donor, while maintaining the antigenic potential of the pathogen to be removed by the recipient's immune system.

It should be noted that in the drawings, like elements are represented by like numerals.

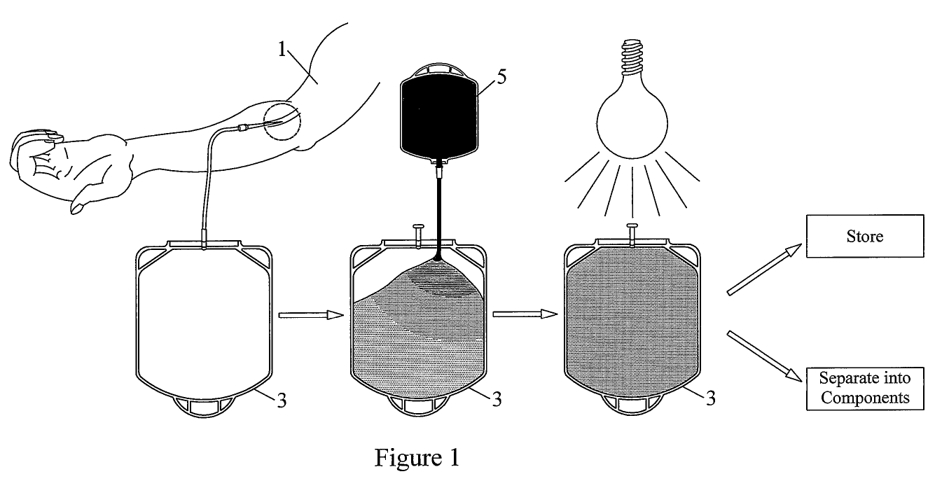

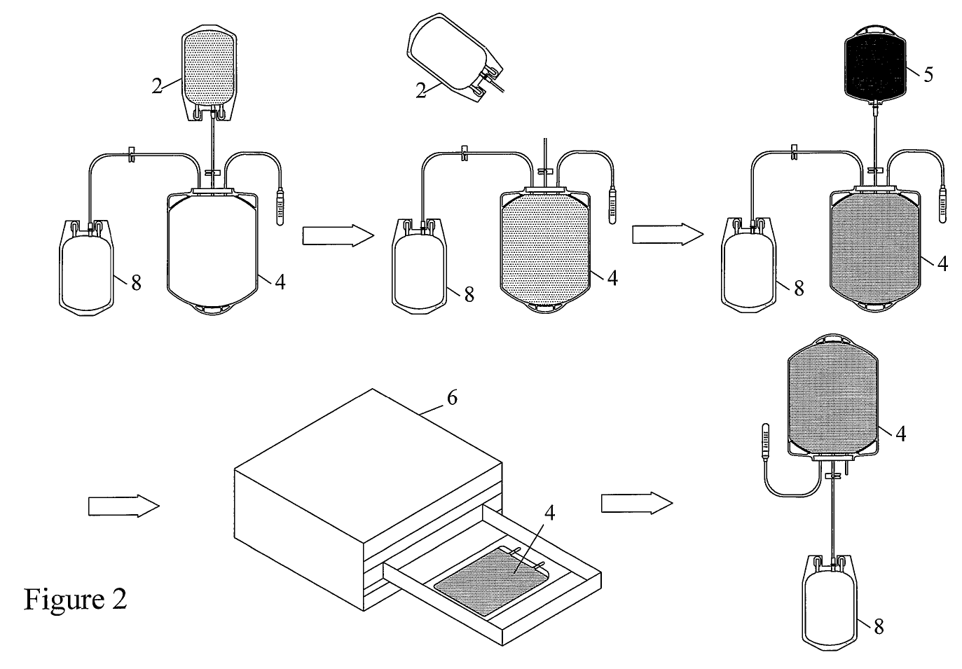

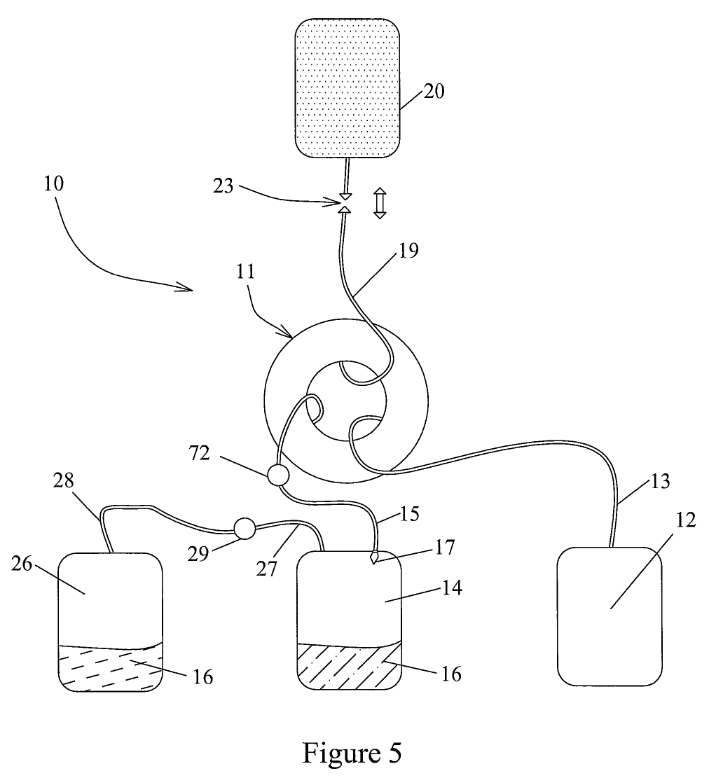

As shown in FIG. 1, whole blood to be pathogen inactivated may be collected from a donor by any method known in the art. Typically, a unit of whole blood (450 mL) is collected from a donor 1 into a whole blood collection bag 3. The collection bag may be a standard blood collection bag (shown in FIG. 1), or may be a round bag such as bag 11 shown in FIG. 5. Once the whole blood is collected, the blood can be transferred to a separate illumination bag (see FIG. 2), or can be illuminated in the collection bag 3, depending upon the material of the collection bag. If illumination is to take place in the collection bag, the collection bag must be at least light permeable and of a size that permits mixing of the whole blood and photosensitizer during illumination. 35 mL of 500 μM riboflavin contained in a bag 5 is added to the whole blood in bag 3, and the whole blood+riboflavin in bag 3 is illuminated with between 22-110 J/mL_{RBC} of radiation. After illumination, the inactivated whole blood can either be stored for later use or can be separated into desired components which may either be used immediately or stored for later use. The pathogen inactivated whole blood may be stored for less than one half hour, or may be stored for a time period up to the point the blood is no longer viable. With the present invention, whole blood does not need to be leukoreduced before the addition of photosensitizer, illumination and subsequent pathogen inactivation, nor does the whole blood or separated pathogen reduced blood components need to be leukoreduced at any time before infusion into a patient.

In another embodiment shown in FIG. 2, whole blood is collected in a whole blood collection bag 2 and transferred to an illumination bag 4. Bag 2 is subsequently removed from the remaining tubing set. Photosensitizer 5 is then added to the whole blood in the illumination bag 4 and the bag is illuminated in an illuminator 6. After illumination, the pathogen inactivated whole blood may be transferred to an integrally attached or pre-connected storage bag 8.

If it is desired to separate the inactivated whole blood into various blood components, the inactivated whole blood can be separated manually or using an automated whole blood separator.

Whole blood is most commonly separated into components manually. After whole blood is collected from a patient, the whole blood is processed in a laboratory. In the processing laboratory, a technician places the bags of whole blood into a large, swinging bucket centrifuge, which must be carefully balanced as the bags are loaded. The centrifuge is started and the bags are spun at a high rate of speed. In the first centrifugation, the red cells, which are the densest component, are forced to the bottom of the bag while the platelet-rich plasma, which is lighter, rises to the top.

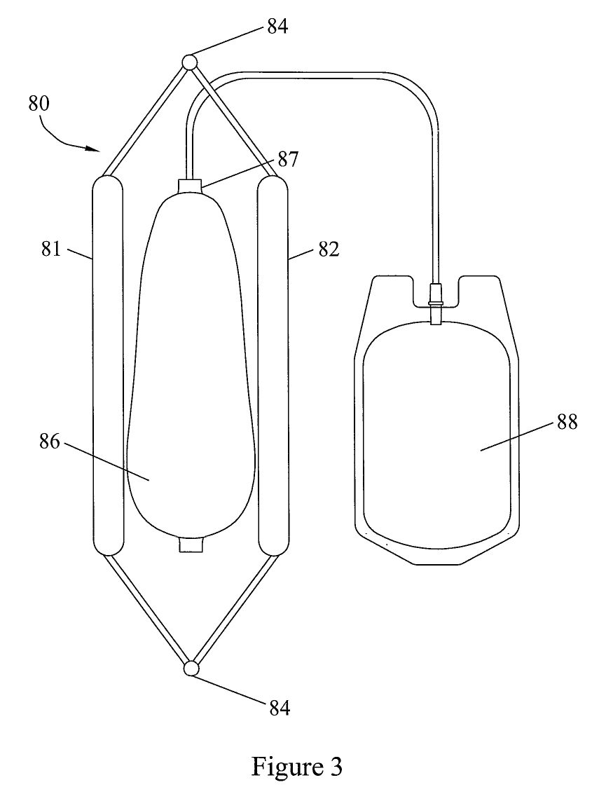

The technician next places each bag in an expresser 80 (see FIG. 3) consisting of two rigid plates 81, 82 that are joined by a spring loaded hinge 84. One of the plates is fixed and the other is moveable. The blood bag 86 is positioned between the two plates and the spring catch released causing the moveable plate to press against the bag. A port 87 on the top of the bag is then opened and the platelet-rich plasma is expressed into a pre-connected, empty bag 88. When the technician observes that red cells are about to reach the outlet port, the expression is stopped and the tubing clamped.

If platelets are to be separated, the bags containing the platelet rich plasma are returned to the centrifuge, the rotor is again balanced and a second spin begins, this time at a higher speed. This spin forces the platelets to the bottom of the bag and allows the lighter plasma to rise to the top. The expression process described above is then repeated so that the platelets can be diverted to a separate bag for storage.

Whole blood may also be separated into components using an automated whole blood separator. Whole blood separation can be performed to obtain either two component products, for example plasma and red blood cells (RBCs), or to obtain three (or more) component products, for example plasma, RBCs and either a buffy coat or a platelet product. The present system and method may be desirable particularly when pathogen reduced whole blood is to be separated into components in a completely sterile manner.

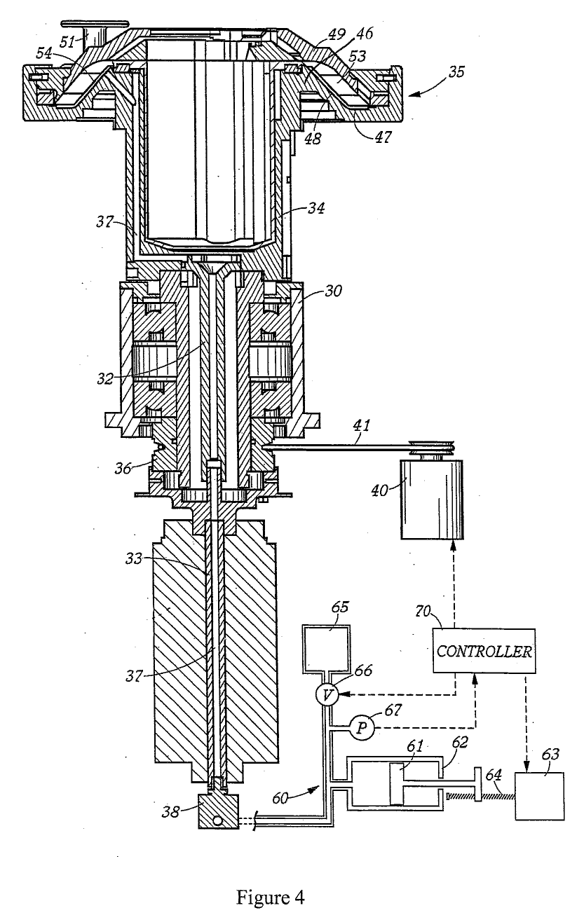

FIG. 4 shows an embodiment of an apparatus for separating a volume of composite liquid by centrifugation. The apparatus comprises a centrifuge adapted for receiving the separation bags shown in FIG. 5 and a component transferring means for causing the transfer of separated components into the satellite bags.

The centrifuge comprises a rotor that is supported by a bearing assembly 30 allowing the rotor to rotate about a vertical central axis (not shown). The rotor includes a cylindrical rotor shaft 32, 33; a central compartment 34 for containing satellite bags, which is connected to the rotor shaft 32, 33; a support member (not shown in FIG. 4) for supporting at least one satellite bag in a determined position within the central compartment 34; and a circular turntable 35 for supporting a separation bag, which is connected to compartment 34.

The rotor shaft comprises a first upper portion 32 and a second lower portion 33. The upper portion 32 of the shaft extends in part through the bearing assembly 30. A pulley 36 is connected to the lower end of the upper portion 32 of the shaft.

The centrifuge further comprises a motor 40 coupled to the rotor by a belt 41 engaged in a groove of the pulley 36 so as to rotate the rotor about a central vertical axis.

The separation apparatus further comprises a first, second and third pinch valve members (not shown) that are mounted on the rotor for selectively blocking or allowing a flow of liquid through a flexible plastic tube, and selectively sealing and cutting a plastic tube.

The turntable 35 comprises a central frusto-conical portion 46, the upper, smaller edge of which is connected to the rim of the compartment 34, an annular flat portion 47 connected to the lower, larger edge of the frusto-conical portion 46, and an outer cylindrical flange 48 extending upwards from the outer periphery of the annular portion 47. The turntable 35 further comprises a vaulted circular lid 49 that is secured to the flange 48 by a hinge so as to pivot between an open and a closed position. The lid 49 is fitted with a lock 51 by which it can be blocked in the closed position. The lid 49 comprises a large cut-out in its upper part that gives access to the central compartment 34 of the rotor. The lid 49 has an annular interior surface that is so shaped that, when the lid 49 is in the closed position, it defines with the frusto-conical portion 46 and the annular flat portion 47 of the turntable 38 a frusto-conical annular compartment 53 having a radial cross-section that has substantially the shape of a parallelogram. The frusto-conical annular compartment 53, later the “separation compartment”, is intended for containing the separation bag 11.

The component transferring means comprises a squeezing system for squeezing the separation bag within the separation compartment 53 and causing the transfer of separated components into the satellite bags. The squeezing system comprises a flexible annular diaphragm 54 that is so shaped as to line the frusto-conical portion 46 and the annular flat portion 47 of the turntable 35, to which it is secured along its smaller and larger circular edges. The squeezing system further comprises a hydraulic pumping station 60 for pumping a hydraulic liquid in and out an expandable hydraulic chamber defined between the flexible diaphragm 54 and the turntable 35, via a duct 37 extending through the rotor from the lower end of the lower portion 33 of the rotor shaft to the turntable 35. The pumping station 60 comprises a piston pump having a piston 61 movable in a hydraulic cylinder 62 fluidly connected via a rotary fluid coupling 38 to the rotor duct 37. The piston 61 is actuated by a stepper motor 63 that moves a lead screw 64 linked to the piston rod. The hydraulic cylinder 62 is also connected to a hydraulic liquid reservoir 65 having an access controlled by a valve 66 for selectively allowing the introduction or the withdrawal of hydraulic liquid into and from a hydraulic circuit including the hydraulic cylinder 62, the rotor duct 37 and the expandable hydraulic chamber. A pressure gauge 67 is connected to the hydraulic circuit for measuring the hydraulic pressure therein.

The separation apparatus further comprises a controller 70 including a control unit (microprocessor) and a memory for providing the microprocessor with information and programmed instructions relative to various separation protocols and to the operation of the apparatus in accordance with such separation protocols. In particular, the microprocessor is programmed for receiving information relative to the centrifugation speed(s) at which the rotor is to be rotated during the various stages of a separation process, and information relative to the various transfer flow rates at which separated components are to be transferred from the separation bag 11 into the satellite bags 12, 14. The information relative to the various transfer flow rates can be expressed, for example, as hydraulic liquid flow rates in the hydraulic circuit, or as rotation speeds of the stepper motor 63 of the hydraulic pumping station 60. The microprocessor is further programmed for receiving, directly or through the memory, information from the pressure gauge 67 and from the photocells (not shown) and for controlling the centrifuge motor 40, the stepper motor 63, and the pinch valve members so as to cause the separation apparatus to operate along a selected separation protocol.

A variety of alternative sets 10 of containers which may be used with the system/machine of the present invention are shown in FIG. 5. A separation container 11 may be a part of the bag set wherein in an embodiment, the separation container 11 is annular and/or of a ring shape. In some embodiments this may be flat, or it may be a somewhat frusto-conical separation container 11 and may be of a flexible plastic material, which in some instances, may be of the same or a similar type as used in conventional blood or blood component or other biological fluid bags. If bag 11 is to be illuminated, it must also be at least light permeable.

As shown in the substantially schematic embodiment of FIG. 5, a first component collection container 12 may be connected by a tube 13 to the separation container 11, and a second component collection container 14 may similarly be connected to the separation container 11 by a second tube 15. Both such connections may be at the inner circumference of ring bag 11 or though not shown, either or both could be connected to the outer circumference or at any desired radial location therebetween. The component collection containers 12, 14 may be shaped in any of a variety of ways and/or formed of any of a variety of materials, though they may in some embodiments be, as shown, substantially rectangular bags of flexible plastic sheet material of substantially conventional type, the plastic sheet material being preferably selected with a view to the type of cells or blood component products which may be chosen to be stored in the respective container. In the two component (2C) set 10 of FIG. 4, these two component collection bags 12, 14 are the only end component bags; however, in the three component (3C) set, a third component collection bag (not shown) may also be connected by a third tubing line, to the ring bag 11. In pathogen inactivated whole blood (WB) separation, the first collection bag 12 may be adapted to receive plasma, the second collection bag 14 adapted to receive RBCs and the third collection bag (in the 3C set), adapted to receive platelets.

In the separation of pathogen inactivated whole blood and the preparation of pathogen inactivated blood components, the bags may all be initially empty, or one or more of the finished component collection container or product bags, e.g., the second component container 14 (in FIG. 5) may be initially filled with a certain amount of an additive or storage fluid or liquid 16 for the component to be disposed therein, e.g., red blood cells. Examples of such a fluid may include storage solutions such as SAG, SAG-M, AS-1, AS-3, or AS-5.

As an alternative, the storage or additive solution may be predisposed in an optional separate bag, see, for example, satellite bag 26 in FIG. 5, which would be connected to or connectable with bag 14 via an additive solution tube 27 leading from bag 14, and a connecting tube 28. An optional sterile barrier or filter 29 represented schematically on line 28 may also be included. The additive or storage solution 16 may then be passed from such a satellite container 26 to container 14 via lines 28, 27. In some embodiments, the solution bag 26 may be pre-connected to bag 14, i.e., during the manufacturing process of the set 10, or as an alternative, the additive solution bag 26 may be later connected or docked via sterile docking or spike connection and thus not be previously stored within or as part of set 10, but instead added at a different time, before or after blood component separation/processing. The component container 14 may in such a case then be temporarily sealed by, for instance, a frangible or a breaking pin 17, or other sealing means such as a peelable or pressure rupturable seal, to keep the solution sealed therein until its use may be desired, i.e., until loaded in the centrifuge and ready to receive a component product such as RBCs.

With the other component bag(s), a storage or an additive solution may similarly be pre-disposed in or adapted to be added to the bag(s) for the benefit of the component product to be later added thereto. If platelets are to be collected, the third component collection container may contain a storage solution for platelets such as PAS, PAS IIIM, or Composol.

In an embodiment such as the one illustrated, the separation container 11 may be provided with a connection tube 19 which may be connected by sterile docking 23 to a source of pathogen inactivated whole blood 20. In another embodiment, whole blood may be collected and pathogen inactivated in separation container 11, in accordance with the principles described in FIGS. 1 and 2 above. In this embodiment, there would be no need for bag 20 and tubing line 19.

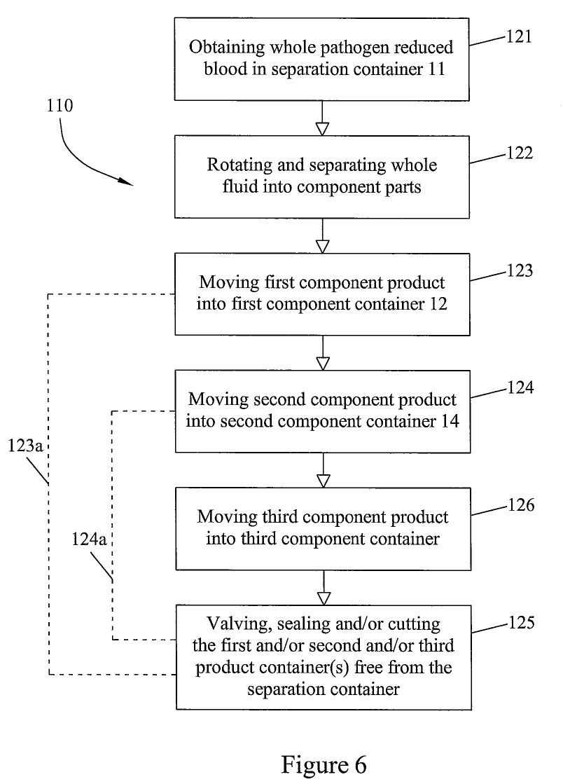

As shown in FIG. 6 in a first step 121 of the general process 110, the pathogen inactivated whole blood is supplied to the separation container/bag 11. Then, in a second general step 122, the pathogen inactivated whole blood is spun and the component parts thereby separated. Next, as shown in box 123, a first component product is moved or expressed out of the separation container 11 to a first component container 12. The second component product is also moved or expressed out of the separation container 11 to its second component container 14. This is depicted by box 124 in the process diagram 110. Lastly, the first and/or second component containers 12, 14 are closed off by valving, sealing and/or cutting the inlets, e.g., tubing lines, thereto. This is depicted by/in box 125. Note, as a general concept, the third, fourth and fifth steps 123, 124, and 125 may occur independently and/or after a decrease in rotation speed of the centrifuge and separation of the second step 122, or more generally here, the rotation/centrifugation of step 122 continues throughout the performance of the other steps 123, 124 and/or 125 and any alternatives and/or intermediary steps thereto. Thus, the rotation/centrifugation and separation step 122 will most often here, cease usually only after completion of steps 123, 124 and/or 125 and any intermediaries and/or alternatives thereto. Cessations of the second step 122 would then constitute the end of the usual process (note, unloading and/or other administrative-type handling processes, marking, labeling, storing and the like post centrifugation process steps, if performed post-processing, notwithstanding). Note, an alternative, optional process line 123a is also shown (in dashed lines) in FIG. 5 to emphasize the alternative that a valving, sealing and/or cutting step 125 may be performed relative to the first component container prior to or during the fourth step 124 and, in any event, prior to and separate from the valving, sealing and/or cutting step for the second product container.

If a third product is collected, an intermediate step 126 may be used for the third product movement or expression from the separation container to the third product container. Note, an alternative, optional process line 124a is also shown (in dashed lines) to emphasize the alternative that a valving, sealing and/or cutting step 125 may be performed relative to the second component container prior to or during the intermediate optional step 126 and, in any event, prior to and separate from the valving, sealing and/or cutting step for the third product container.

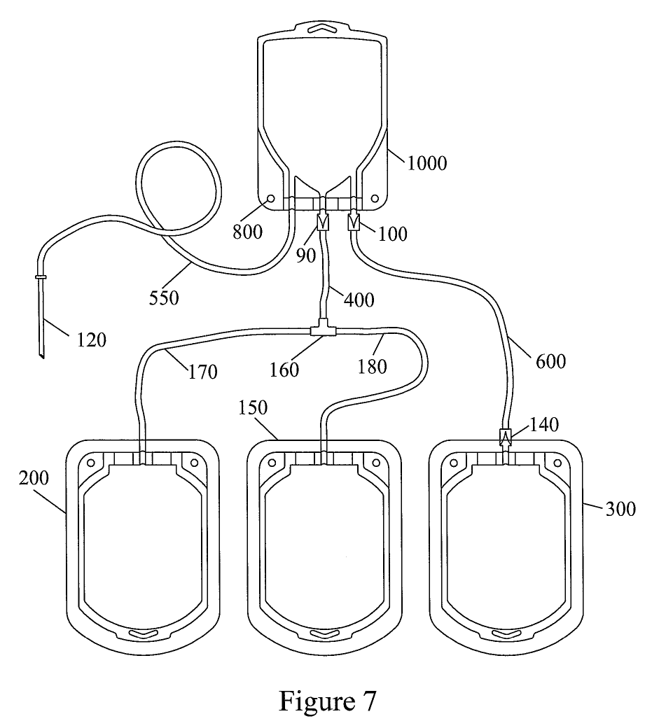

FIG. 7 shows another example of a set of bags adapted for another system/machine which may be used in centrifugal separation of pathogen inactivated whole blood into component products. This bag set comprises a flexible separation bag 1000 and three flexible satellite bags 200, 300, 150 connected thereto.

The separation bag 1000 may be used as a whole blood collection bag, a pathogen inactivation bag and a bag for separating the pathogen inactivated whole blood into components. The separation bag 1000 is flat and generally rectangular. It is made of two rectangular sheets of plastic material that are welded together so as to define therebetween an interior space having a main rectangular portion connected to a triangular top downstream portion. A first tube 400 is connected to the tip of the triangular portion, and second and third tubes 500, 600 are connected to either lateral edges of the triangular portion, respectively. The proximal ends of the three tubes 400, 500, 600 are embedded between the two sheets of plastic material so as to be parallel. The separation bag 1000 further comprises a hole 800 in each of its corners that are adjacent to the three tubes 400, 500, 600. The holes 800 are used to secure the separation bag to a separation compartment.

A volume of anticoagulant (typically about 63 ml for a blood donation of about 450 ml) is initially added to the separation bag, and the first and third tubes 400, 600 are fitted at their proximal end with a breakable stopper 90, 100 respectively, blocking a liquid flow therethrough.

The second tube 500 is a collection tube having a needle 120 connected to its distal end. At the beginning of a blood donation, the needle 120 is inserted in the vein of a donor and blood flows into the collection (separation) bag 1000. After a desired volume of blood has been collected in the collection (separation) bag 1000, the collection tube 500 is sealed and cut. Photosensitizer may be initially added to bag 1000 before the whole blood is added, or may be added after the whole blood is added through tubing 500. It may also be added through a separate tube (not shown).

The first satellite bag 200 is intended for receiving a plasma component. It is flat and substantially rectangular. It is connected to the distal end of the first tube 400.

The second satellite bag 300 is intended for receiving a red blood cell component. It is flat and substantially rectangular. It is connected to the distal end of the third tube 600. The second satellite bag 300 may contain a volume of storage solution for storage of red blood cells, and the third tube 600 is fitted at its distal end with a breakable stopper 140 blocking liquid flow therethrough.

The third satellite bag 150 is intended to receive a platelet component. Like the first and second satellite bags 200, 300, the third satellite bag 150 is flat and substantially rectangular.

The bag set also contains a T-shaped three-way connector 160 having its leg connected by the first tube 400 to the separation bag 1000, a first arm connected by a fourth tube 170 to the first satellite bag 200 (plasma component bag), and a second arm connected by a fifth tube 180 to the third satellite bag 150 (platelet component bag).

An apparatus for simultaneously separating by centrifugation four discrete volumes of pathogen inactivated whole blood may be used with the bag set of FIG. 7. The apparatus includes a centrifuge adapted to receive the four bag sets shown in FIG. 7, with the four discrete volumes of a composite liquid contained in the four separation bags; a component transferring system for transferring at least one separated component from each separation bag into a satellite bag connected thereto; a first balancing system for initially balancing the rotor when the weights of the four separation bags are different; and a second balancing system for balancing the rotor when the weights of the separated components transferred into the satellite bags cause an unbalance of the rotor.

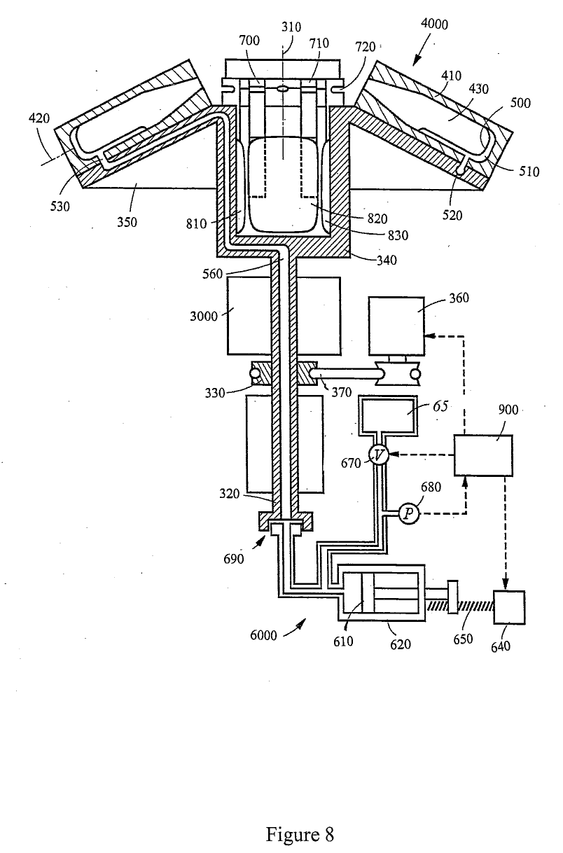

As shown in FIG. 8, the centrifuge comprises a rotor that is supported by a bearing assembly 3000 allowing the rotor to rotate around a rotation axis 310. The rotor includes a cylindrical rotor shaft 320 to which a pulley 330 is connected; a system comprising a central cylindrical container 340 for containing satellite bags, which is connected to the rotor shaft 320 at the upper end thereof so that the longitudinal axis of the rotor shaft 320 and the longitudinal axis of the container 340 coincide with the rotation axis 310, and a frusto-conical turntable 350 connected to the upper part of the central container 340 so that its central axis coincides with the rotation axis 310. The frusto-conical turntable 350 flares underneath the opening of the container 340. Four identical separation cells 4000 are mounted on the turntable 350 so as to form a symmetrical arrangement with respect to the rotation axis 310.

The centrifuge further comprises a motor 360 coupled to the rotor by a belt 370 engaged in a groove of the pulley 330 so as to rotate the rotor about the rotation axis 310.

Each separation cell 4000 comprises a container 410 having the general shape of a rectangular parallelepiped. The separation cells 4000 are mounted on the turntable 350 so that their respective median longitudinal axes 420 intersect the rotation axis 310, so that they are located substantially at the same distance from the rotation axis 310, and so that the angles between their median longitudinal axes 420 are substantially the same (i.e. 90 degrees). The exact position of the separation cells 4000 on the turntable 350 is adjusted so that the weight on the turntable is equally distributed when the separation cells 4000 are empty, i.e. so that the rotor is balanced. It results from the arrangement of the separating cells 4000 on the turntable 350 that the separating cells 4000 are inclined with respect to the rotation axis 310 of an acute angle equal to the angle of the frustum of a cone that geometrically defines the turntable 350.

Each container 410 comprises a cavity 430 that is so shaped and dimensioned as to loosely accommodate a separation bag 1000 full of liquid, of the type shown in FIG. 4. The cavity 430 (which will be referred to later also as the “separation compartment”) is defined by a bottom wall that is the farthest to the rotation axis 310, a lower wall that is the closest to the turntable 350, an upper wall opposite to the lower wall, and two lateral walls. The cavity 430 comprises a main part, extending from the bottom wall, which has substantially the shape of a rectangular parallelepiped with rounded angles, and an upper part, which has substantially the shape of a prism having convergent triangular bases. In other words, the upper part of the cavity 430 is defined by two couples of opposite walls converging towards the central median axis 420 of the cavity 430.

One interest of this design is to cause a radial dilatation of the thin layer of a minor component of whole blood (e.g. the platelets) after separation by centrifugation, and makes it more easily detectable in the upper part of a separation bag. As shown in FIG. 8, the two couples of opposite walls of the upper part of the separation cell 4000 converge towards three cylindrical parallel channels 440, 450, 460, opening at the top of the container 410, and in which, when a separation bag 1000 is set in the container 410, the three tubes 400, 500, 600 extend.

The container 410 also comprises a hinged lateral lid (not shown), which is comprised of an upper portion of the external wall of the container 410, i.e. the wall that is opposite to the turntable 350. The lid is so dimensioned as to allow, when open, an easy loading of a separation bag 1000 full of liquid into the separation cell 4000. The container 410 comprises a fast locking means (not shown) by which the lid can be locked to the remaining part of the container 410.

The container 410 also comprises a securing means for securing a separation bag 1000 within the separation cell 4000. The bag securing means comprises two pins (not shown) protruding on the internal surface of the lid, close to the top of separation cell 4000, and two corresponding recesses in the upper part of the container 410. The two pins are so spaced apart and dimensioned as to fit into the two holes 800 in the upper corner of a separation bag 1000.

The separation apparatus further comprises a component transferring means for transferring at least one separated component from each separation bag into a satellite bag connected thereto. The component transferring means comprises a squeezing system for squeezing the separation bags 1000 within the separation compartments 430 and causing the transfer of separated components into satellite bags 200, 300, 150.

The squeezing system comprises a flexible diaphragm 500 that is secured to each container 410 so as to define an expandable chamber 510 in the cavity thereof. More specifically, the diaphragm 500 is dimensioned so as to line the bottom wall of the cavity 430 and a large portion of the lower wall of the cavity 430, which is the closest to the turntable 350.

The squeezing system further comprises a peripheral circular manifold 520 that forms a ring within the turntable 350 extending close to the periphery of the turntable 350. Each expansion chamber 510 is connected to the manifold 520 by a supply channel 530 that extends through the wall of the respective container 410, close to the bottom thereof.

The squeezing system further comprises a hydraulic pumping station 6000 for pumping a hydraulic liquid in and out the expandable chambers 510 within the separation cells 4000. The hydraulic liquid is selected so as to have a density slightly higher than the density of the more dense of the components in the composite liquid to be separated (e.g. the red blood cells, when the composite liquid is blood). As a result, during centrifugation, the hydraulic liquid within the expandable chambers 510, whatever the volume thereof, will generally remain in the most external part of the separation cells 4000. The pumping station 6000 is connected to the expandable chambers 510, through a rotary seal 690, by a duct 560 that extends through the rotor shaft 320, the bottom and lateral wall of the central container 340, and, from the rim of the central container 340, radially through the turntable 350 where it connects to the manifold 520.

The pumping station 6000 comprises a piston pump having a piston 610 movable in a hydraulic cylinder 620 fluidly connected via a rotary fluid coupling to the rotor duct 540. The piston 610 is actuated by a stepper motor 640 that moves a lead screw 650 linked to the piston rod. The hydraulic cylinder 620 is also connected to a hydraulic liquid reservoir 660 having an access controlled by a valve 670 for selectively allowing the introduction or the withdrawal of hydraulic liquid into and from a hydraulic circuit including the hydraulic cylinder 620, the rotor duct 560 and the expandable hydraulic chambers 510. A pressure gauge 680 is connected to the hydraulic circuit for measuring the hydraulic pressure therein.

The separation apparatus further comprises four pairs of first and second pinch valve members 700, 710 that are mounted on the rotor around the opening of the central container 340. Each pair of pinch valve members 700, 710 faces one separation cell 4000, with which it is associated. The pinch valve members 700, 710 are designed for selectively blocking or allowing a flow of liquid through a flexible plastic tube, and selectively sealing and cutting a plastic tube. Each pinch valve member 700, 710 comprises an elongated cylindrical body and a head having a groove 720 that is defined by a stationary upper jaw and a lower jaw movable between an open and a closed position. The groove 720 is so dimensioned that one of the tubes 400, 170, 180 of the bag set shown in FIG. 7 can be snuggly engaged therein when the lower jaw is in the open position. The elongated body contains a mechanism for moving the lower jaw and it is connected to a radio frequency generator that supplies the energy necessary for sealing and cutting a plastic tube. The pinch valve members 700, 710 are mounted inside the central container 340, adjacent the interior surface thereof, so that their longitudinal axes are parallel to the rotation axis 310 and their heads protrude above the rim of the container 340. Electric power is supplied to the pinch valve members 700, 710 through a slip ring array that is mounted around a lower portion of the rotor shaft 320.

The separation apparatus further comprises a first balancing means for initially balancing the rotor when the weights of the four separation bags 1000 contained in the separation cells 4000 are different. The first balancing means substantially comprises the same structural elements as the elements of the component transferring means described above, namely: four expandable hydraulic chambers 510 interconnected by a peripheral circular manifold 520, and a hydraulic liquid pumping station 6000 for pumping hydraulic liquid into the hydraulic chambers 510 through a rotor duct 560, which is connected to the circular manifold 520. In order to initially balance the rotor, whose four separation cells 4000 contain four discrete volumes of a composite liquid that may not have the same weight (because the four volumes may be not equal, and/or the density of the liquid may slightly differ from one volume to the other one), the pumping station 6000 is controlled so as to pump into the interconnected hydraulic chambers 510, at the onset of a separation process, a predetermined volume of hydraulic liquid that is so selected as to balance the rotor in the most unbalanced situation. For pathogen inactivated whole blood, the determination of this balancing volume takes into account the maximum difference in volume between two blood donations, and the maximum difference in hematocrit (i.e. in density) between two blood donations. Under centrifugation forces, the hydraulic liquid will distribute unevenly in the four separation cells 4000 depending on the difference in weight of the separation bags 1000, and balance the rotor. In order to get an optimal initial balancing, the volume of the cavity 430 of the separation cells 4000 should be selected so that the cavities 430, whatever the volume of the separation bags 1000 contained therein, are not full after the determined amount of hydraulic liquid has been pumped into the interconnected expansion chambers 510.

The separation apparatus further comprises a second balancing means, for balancing the rotor when the weights of the components transferred into the satellite bags 200, 300, 150 in the central container 340 are different. For example, when two blood donations have the same hematocrit and different volumes, the volumes of plasma extracted from each donation are different, and the same is true when two blood donations have the same volume and different hematocrit.

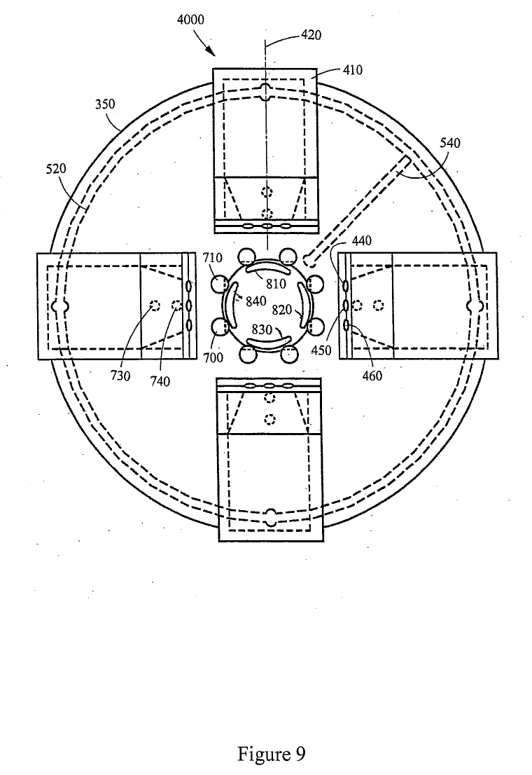

As shown in FIG. 9, the second balancing means comprises four flexible rectangular pouches 810, 820, 830, 840 that are interconnected by four tube sections (not shown), each tube section connecting two adjacent pouches by the bottom thereof. The pouches 810, 820, 830, 840 contain a volume of balancing liquid having a density close to the density of the composite liquid. The volume of balancing liquid is so selected as to balance the rotor in the most unbalanced situation. The four pouches 810, 820, 830, 840 are so dimensioned as to line the inner surface of the central container 340 and to have an internal volume that is larger than the volume of balancing liquid so that the balancing liquid can freely expand in any of the pouches 810, 820, 830, 840. In operation, if, for example, four satellite bags 200 respectively adjacent to the four pouches 810, 820, 830, 840 receive different volumes of a plasma component, the four satellite bags 200 will press unevenly, under centrifugation forces, against the four pouches 810, 820, 830, 840, which will result in the balancing liquid becoming unevenly distributed in the four pouches 810, 820, 830, 840 and compensating for the difference in weight in the satellite bags 200.

The separation apparatus further comprises a controller 900 including a control unit (e.g. a microprocessor) and a memory unit for providing the microprocessor with information and programmed instructions relative to various separation protocols (e.g. a protocol for the separation of a plasma component and a blood cell component, or a protocol for the separation of a plasma component, a platelet component, and a red blood cell component) and to the operation of the apparatus in accordance with such separation protocols. In particular, the microprocessor is programmed for receiving information relative to the centrifugation speed(s) at which the rotor is to be rotated during the various stages of a separation process (e.g. stage of component separation, stage of a plasma component expression, stage of suspension of platelets in a plasma fraction, stage of a platelet component expression, etc), and information relative to the various transfer flow rates at which separated components are to be transferred from the separation bag 1000 into the satellite bags 200, 300, 150. The information relative to the various transfer flow rates can be expressed, for example, as hydraulic liquid flow rates in the hydraulic circuit, or as rotation speeds of the stepper motor 640 of the hydraulic pumping station 6000. The microprocessor is further programmed for receiving, directly or through the memory, information from the pressure gauge 680 and from the four pairs of photocells 730, 740 and for controlling the centrifuge motor 360, the stepper motor 640 of the pumping station 6000, and the four pairs of pinch valve members 700, 710 so as to cause the separation apparatus to operate along a selected separation protocol.

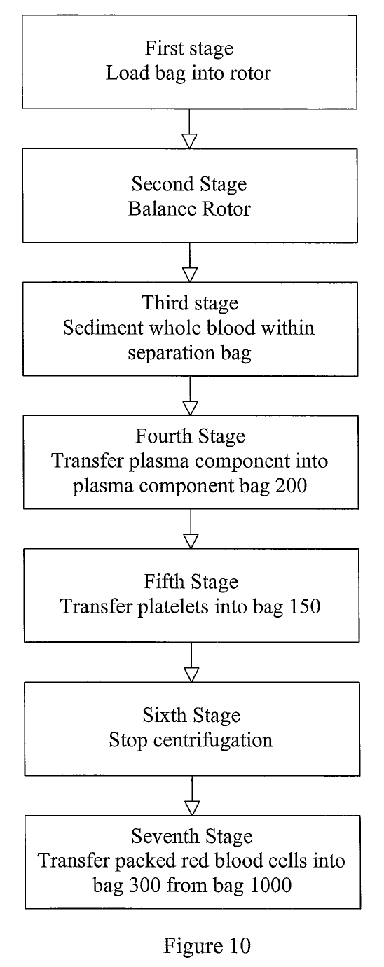

According to the separation protocol shown in FIG. 10, four discrete volumes of pathogen reduced blood are separated into a plasma component, a first cell component comprising platelets, white blood cells, some red blood cells and a small volume of plasma (later the “buffy coat” component) and a second cell component mainly comprising red blood cells. Each volume of blood is contained in a separation bag 1000 of a bag set represented in FIG. 7, in which it has previously been collected from a donor using the collection tube 500. After the blood collection, the collection tube 500 has been sealed and cut close to the separation bag. Typically, the volumes of blood are not the same in the four separation bags 1000, and the hematocrit varies from one separation bag 1000 to another one. Consequently, the separation bags 1000 have slightly different weights.

As shown in FIG. 10, the first stage of the separation procedure begins by loading the four bag sets into the four separation cells 4000 on the rotor.

In the second stage, the rotor is balanced in order to compensate for the difference in weights of the separation bags.

In the third stage, the pathogen inactivated whole blood within the separation bag 1000 is sedimented to a desired level.

In the fourth stage the plasma component is transferred into the plasma component bag 200.

In the fifth stage the platelet component is transferred into the platelet component bag 150.

In the sixth stage the centrifugation process is ended.

When the fifth stage is completed, the red blood cells are transferred from separation bag 1000 into the red blood cell component bag 300.

Pathogen inactivated whole blood may also be separated into blood components using the whole blood separator described in U.S. Pat. No. 6,910,998.

In any of the whole blood separation processes described above, no prior leukoreduction of the pathogen inactivated whole blood before separation into individual components is necessary. Nor is it necessary to leukoreduce the red blood cells after separation. Pathogen inactivation of the whole blood using riboflavin and light functionally inactivates the white blood cells in the whole blood. This is shown in Example 1 below. A pathogen inactivation procedure is particularly important when buffy coats containing white blood cells are collected.

### Methods

For the control units, whole blood is processed manually, centrifuged using a soft spin, the platelet rich plasma (PRP) supernatant expressed, and a full volume (approximately 100 mL) of AS-3 additive solution added to the separated RBCs for storage. A platelet concentrate is made from the PRP and stored at 22° C. in a Helmer incubator for 1 day and 5 days prior to sampling for Day 1 and Day 5 platelet quality measurements. The remaining plasma is stored frozen for 28 days, and protein quality assessed for Day 0 and Day 28 samples. The plasma, platelet concentrates and RBCs for the controls undergo the same testing as the treated units.

For the treated units, 35 mL of riboflavin is added to 470±10 mL of whole blood in a 1 L ELP bag and illuminated at 22, 33, 44, 80 and 110 J/mL_{RBC} in an illuminator (Mirasol® Whole Blood Illuminator R5.0.wb.12, available from CaridianBCT, Inc., Lakewood, Colo.). A sample is removed pre-illumination to measure in vitro plasma quality. After illumination, the whole blood is transferred to a UBB bag, centrifuged using a soft spin, the PRP/riboflavin supernatant expressed, and a full volume bag of AS-3 additive solution (approximately 100 mL) is added to the RBCs for storage. A platelet component and plasma component were made from the PRP as described above. The platelet component is stored at 22° C. in a Helmer incubator prior to sampling for Day 1 and Day 5 platelet quality measurements.

Platelet quality is assessed with measurements of pH, swirl, lactate production rate and glucose consumption rate on Days 1 and 5.

Plasma quality is assessed with measurements of fibrinogen, total protein, and Factors V, VIII and XI on Day 0 and Day 28 of frozen storage.

RBC quality is monitored through Day 42 of storage at 4° C. to assess hemolysis, osmotic fragility and ATP release. Samples were removed for Day 0 sampling with subsequent sampling occurring on Days 28, 35 and 42. Red blood cell quality was also assessed without separation of red blood cells from the whole blood. Treated whole blood was stored at room temperature and percent hemolysis and potassium concentration ([K⁺]) were measured.

### EXAMPLE 1

Transfusion of blood products containing white blood cells (WBC) can result in the induction of immune responses that can negatively impact the transfusion recipient. These immunological consequences can include transfusion-associated graft-versus-host disease (TA-GvHD) and production of cytokines and alloantibodies. TA-GvHD, a donor-anti-recipient response, is almost always fatal and is mediated by proliferating T lymphocytes of the donor. The standard approach to inactivate leukocytes and prevent TA-GvHD has been to expose blood products to γ-irradiation.

In the following assays, non-leukoreduced units of fresh (<8 hours from collection) whole blood were treated at energies of 22, 33 and 44 J/mL_{RBC}. For treated cells, riboflavin was added to the whole blood before illumination. After illumination, leukocytes were isolated from the whole blood units and the functionality of white blood cells (WBCs) was assessed for: (1) exhibiting cell activation (CD69 expression) in response to PMA (Phorbol 12-myristate 13-acetate), (2) WBC proliferation in response to PHA (Phytohemagglutinin), to allogeneic stimulating cells, and to CD3/CD28 stimulation, (3) antigen presentation to allogeneic responder cells, and (4) the ability of WBCs to produce cytokines in response to LPS (lipopolysaccharide) or CD3/CD28 antibodies.

### 1.) The Ability of WBCs to be Activated by PMA and Express CD69

CD69 is an early activation marker on T cells and can be visualized by flow cytometry using anti-CD69 antibodies. Within 4 hrs of T-cell activation, CD69 is detectable and stays upregulated as long as the cell is in an activated state. As shown in Graph 1, treatment with riboflavin and light inhibited expression of CD69 on T cells after PMA activation at all energies tested.

2.) WBC Proliferation in Response to PHA and anti-CD3/CD28

The ability of treated WBCs to proliferate was analyzed by thymidine incorporation after 3 days of incubation. As shown in Graph 2, exposure to PHA (A) or to plate-bound anti-CD3 plus anti-CD28 antibodies (B) induced proliferation in untreated WBCs. Treated WBCs showed no detectable induced proliferation at 33 J/ml_{RBC} and above when exposed to these mitogens.

### 3.) WBC Proliferation in Response to Allogeneic Stimulators and Antigen Presentation to Allogeneic Responder Cells

WBCs in blood products are able to present antigen to recipient cells and induce proliferation and allo-antibody formation. Treated WBCs were evaluated in Mixed Lymphocyte culture (MLC) both as responder cells (proliferate in response to stimulation) and as stimulators (promote proliferation of responder WBCs). Treated WBCs tested as responder cells in the MLC were not able to proliferate in response to allogeneic stimulator cells (Graph 3A), but untreated WBCs were. The amount of proliferation detected in a MLC depends on the stimulator-responder combination, and thus is donor dependent. Allogeneic stimulator cells were treated with mitomycin C (a mitotic spindle poison) to prevent proliferation of the stimulator cells in culture with untreated and treated responder cells.

Treated WBCs (stimulators) were analyzed for their ability to induce proliferation of allogeneic responder cells (Graph 3B) in the MLC. No proliferation by allogeneic responder cells could be detected, indicating that treatment with riboflavin and light inhibits antigen presentation of WBCs.

In summary, untreated WBCs proliferated in response to mitogens (PHA), surface receptor crosslinking by antibodies (anti-CD3/CD28) and allogeneic stimulator cells. In contrast, treatment of WBCs with riboflavin+UV light at all energies tested inhibited proliferation in response to any of these stimuli, showing that antigen specific as well as unspecific induction of proliferation is blocked due to treatment. Treated WBCs did not present antigen or induce proliferation in allogeneic responder cells, while untreated WBCs did.

Levels of inhibition of proliferation due to treatment are shown in Table 1, comparing stimulated control cells to stimulated treated cells. The proliferative response was decreased 93-99%. The proliferation of treated cells is down to detection limits of the assay, since levels of proliferation of stimulated treated cells are as low as proliferation levels detected in cells cultured in PBS. A comparison of stimulated control cells to PBS cultured control cells shows a decrease in proliferation of 99% (data not shown).

TABLE 1
Levels of inhibition

| | % inhibition | | |
|---|---|---|---|
| | 22 J/ | | |
| | ml_{RBC} | 33 J/ml_{RBC} | 44 J/ml_{RBC} |
| PHA | 98 ± 1 | 99 ± 1 | 99 ± 0 |
| CD3/28 | 93 ± 2 | 99 ± 0 | 99 ± 0 |
| Allorecognition: treated responders | 95 ± 3 | 95 ± 2 | 96 ± 2 |
| Allostimulation: treated stimulators | 93 ± 3 | 96 ± 2 | 95 ± 2 |

### 4.) Ability of WBCs to Produce Cytokines in Response to Anti-CD3/CD28 or LPS.

Another measure of functionality of WBCs is to measure cytokine production in response to LPS (Lipopolysaccharide) or anti-CD3/CD28 antibodies. Stimulation with anti-CD3/28 induces cytokine production in T cells (TH1/TH2 cytokines). LPS activates monocytes and macrophages leading to the release of inflammatory cytokines. Cytokines were detected using CBA ((Cytometric Bead Assay) (kits purchased from BD Biosciences, PharMingen). A solution with standards is provided in the kit. Based on the values obtained for the standard curve a computer program determines a linear regression and the results of the individual samples. The limit of detection of these CBA assays is approximately 5-10 pg/ml.

As shown in Table 2A, the induction of TH1/TH2 cytokines is higher with anti-CD3/28 stimulation (Table 2A) than with LPS (Table 2B). Treatment significantly reduced TH1/TH2 cytokine production induced by anti-CD3/28 stimulation to levels comparable to the medium control of treated or untreated cells at all energies tested. When exposed to anti-CD3/28 antibodies, IL-2, TNF-α and IFN-γ production was not reduced to medium control levels at 33 J/ml_{RBC} and above, but compared to cytokine levels produced by untreated cells, the level of cytokine production was inhibited by >90%. TH1/TH2 cytokine levels induced by LPS were reduced to medium control levels after treatment at 44 J/ml_{RBC}.

Inflammatory cytokines are induced by LPS as well as anti-CD3/28 stimulation. Treatment significantly reduced inflammatory cytokine production in response to anti-CD3/28 antibodies. High levels of IL-8 in the medium control represent stored cytokines, rather than produced cytokines. Treatment also reduced the levels of IL-8 in medium control cells. Inflammatory cytokine production in response to LPS was reduced with treatment, but not to medium control levels as seen with anti-CD3/28 stimulation.

Cytokine production in response to anti-CD3/anti-CD28 antibodies was blocked >90% at all energies tested, with the exception of IL-4 and IL-8 at 22 J/ml_{RBC} (see Table 2A). Inhibition of cytokine production in response to LPS was below 90% at 33 J/ml_{RBC} for the following cytokines: IL-5 and IL-2. IL-5 and IL-2 are produced at very low levels in untreated cells in response to LPS and are reduced to levels of detection after treatment. TNF-α and IL-10 were measured using the CBA kits for inflammatory and TH1/TH2 cytokines.

Standard deviation values were high in samples after LPS stimulation, compared to values obtained after anti-CD3/28 stimulation. Anti-CD3/28 stimulation specifically activates T lymphocytes through the T cell receptor. In contrast, LPS is a major component of the outer membrane of Gram-negative bacteria and promotes the secretion of cytokines in many cell types, mainly macrophages. This endotoxin function of LPS triggering a polyclonal response may explain the observed variability between donors.

TABLE 2
Inhibition of cytokine production after treatment

| | | % inhibition | | |
|---|---|---|---|---|
| | | 22 J/ml_{RBC} | 33 J/ml_{RBC} | 44 J/ml_{RBC} |
| A | | | | |
| anti-CD3/28 | | | | |
| Inflammatory | IL-12 p70 | 98 ± 4 | 99 ± 2 | 97 ± 4 |
| Cytokines | TNFα | 90 ± 12 | 100 ± 0 | 100 ± 0 |
| | IL-10 | 99 ± 1 | 100 ± 0 | 100 ± 0 |
| | IL-6 | 99 ± 2 | 100 ± 0 | 100 ± 0 |
| | IL-1β | 98 ± 2 | 100 ± 0 | 100 ± 0 |
| | IL-8 | 89 ± 5 | 98 ± 2 | 100 ± 0 |
| TH1/TH2 | IFN-γ | 99 ± 1 | 100 ± 0 | 100 ± 0 |
| cytokines | TNFα | 92 ± 15 | 99 ± 1 | 100 ± 0 |
| | IL-5 | 94 ± 3 | 99 ± 1 | 99 ± 1 |
| | IL-4 | 84 ± 11 | 95 ± 3 | 97 ± 2 |
| | IL-2 | 91 ± 7 | 99 ± 1 | 100 ± 0 |
| | IL-10 | 99 ± 1 | 100 ± 0 | 100 ± 0 |
| B | | | | |
| LPS | | | | |
| Inflammatory | IL-12 p70 | NA | NA | NA |
| Cytokines | TNFα | 54 ± 35 | 92 ± 8 | 98 ± 2 |
| | IL-10 | 63 ± 21 | 94 ± 5 | 99 ± 1 |
| | IL-6 | 84 ± 8 | 96 ± 3 | 99 ± 1 |
| | IL-1β | 68 ± 19 | 91 ± 8 | 98 ± 3 |
| | IL-8 | 85 ± 8 | 96 ± 4 | 99 ± 1 |
| TH1/TH2 | IFN-γ | 63 ± 71 | 93 ± 8 | 93 ± 7 |
| cytokines | TNFα | 45 ± 53 | 94 ± 5 | 94 ± 8 |
| | IL-10 | 66 ± 21 | 94 ± 4 | 99 ± 1 |
| | IL-5 | 89 ± 2 | 89 ± 5 | 91 ± 6 |
| | IL-4 | 59 ± 65 | 90 ± 4 | 90 ± 4 |
| | IL-2 | 63 ± 54 | 88 ± 5 | 88 ± 4 |

In summary, WBC proliferation in response to all tested stimuli and antigen presentation to allogeneic responder cells was inhibited >90% at all energies tested. Cytokine production in response to anti-CD3/anti-CD28 antibodies was blocked >90% at all energies tested, with the exception of IL-4 and IL-8 at 22 J/ml_{RBC}, that were inhibited by 84% and 89% respectively.

### EXAMPLE 2

To test whether pathogen inactivation of whole blood was effective in inactivating viruses which may be present in the whole blood, both non-enveloped and enveloped model viruses were tested. Hepatitis A (HAV), canine parvovirus (CPV), vesicular stomatitis virus (VSV) and infectious bovine rhinotracheitis virus (IBR) were the viruses used.

As can be seen in FIG. 10, log reduction of both non-enveloped (FIG. 10A) and enveloped (FIG. 10B) viruses increases linearly with respect to energy.

### EXAMPLE 3

To test whether pathogen inactivation of whole blood was effective in inactivating clinically relevant levels of bacteria which may be present in the whole blood, low titer bacteria studies were done. After pathogen inactivation of whole blood, the whole blood was separated into a red blood cell (RBC) component and platelet rich plasma (PRP) component. The results are shown in Table 3 below. A + symbol means that some of the replicates of the cultures in the panel grew in under 5 days. A − symbol means that none of the replicates of the cultures in the panel grew in under 5 days.

TABLE 3
Low bacteria titer studies

| | Bacteria detected (+) | | | | | |
|---|---|---|---|---|---|---|
| | or not detected (−) after treatment | | | | | |
| | 44 J/mL_{RBC} | | 80 J/mL_{RBC} | | 110 J/mL_{RBC} | |
| | (n = 8) | | (n = 3) | | (n = 3) | |
| | RBC | PRP | RBC | PRP | RBC | PRP |
| S. epidermidis | not | not | +/−^{a} | +/−^{a} | + | − |
| | measured | measured | | | | |
| Y. enterocolitica | not | not | − | − | − | − |
| | measured | measured | | | | |
| S. liquefaciens | +/−^{c} | +/−^{d} | − | − | − | − |
| A. baumannii | +/−^{e} | +/−^{d} | +/−^{b} | +/−^{b} | − | − |
| S. pyogenes | not | not | +/−^{b} | − | − | − |
| | measured | measured | | | | |

^{a}1 of 2 replicates negative
^{b}2 of 3 replicates negative
^{c}7 of 8 replicates negative
^{d}3 of 8 replicates negative
^{e}1 of 8 replicates negative

As seen in Table 3, only S. epidermidis grew in red blood cells illuminated at 110 J/mL_{RBC}.

### EXAMPLE 4

Whole blood was illuminated at 20, 33, 44, 60, 80 and 110 J/mL_{RBC}. The separated red blood cell component was stored at 4° C. up to 42 days in AS-3. The percentage of red blood cell hemolysis during storage was measured after 28, 35 and 42 days of storage.

As shown in FIG. 11, whole blood illuminated at 110 J/mL_{RBC} had the greatest amount of hemolysis at 42 days of storage. Illumination at the other energies did not produce significant differences in hemolysis over 42 days of storage.

### EXAMPLE 5

ATP level or concentration was measured in red blood cells separated from pathogen inactivated whole blood. ATP concentration is a measure of the amount of ATP present in the cells at a given time.

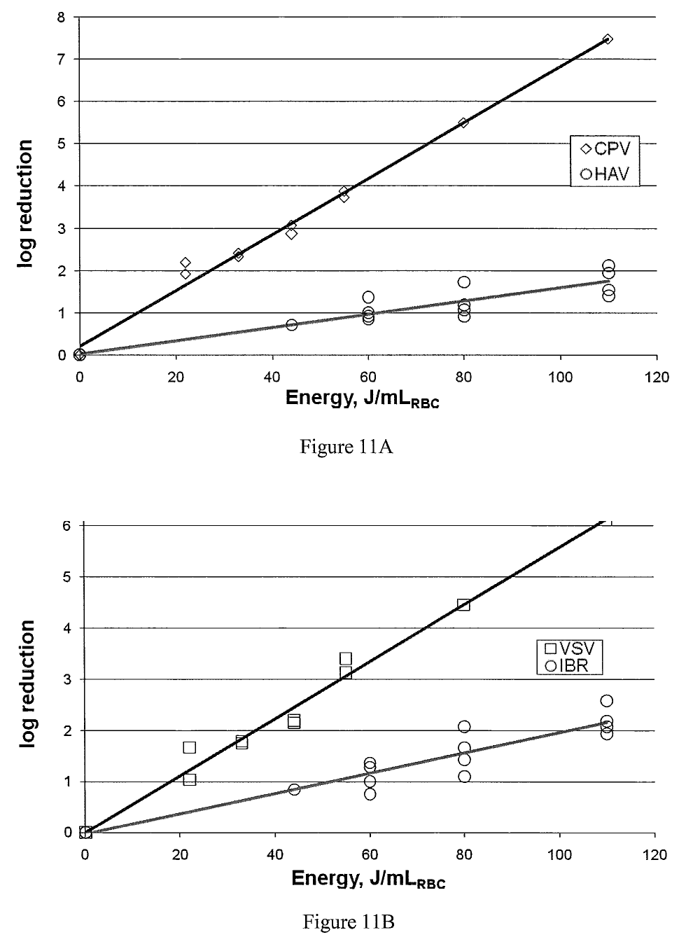

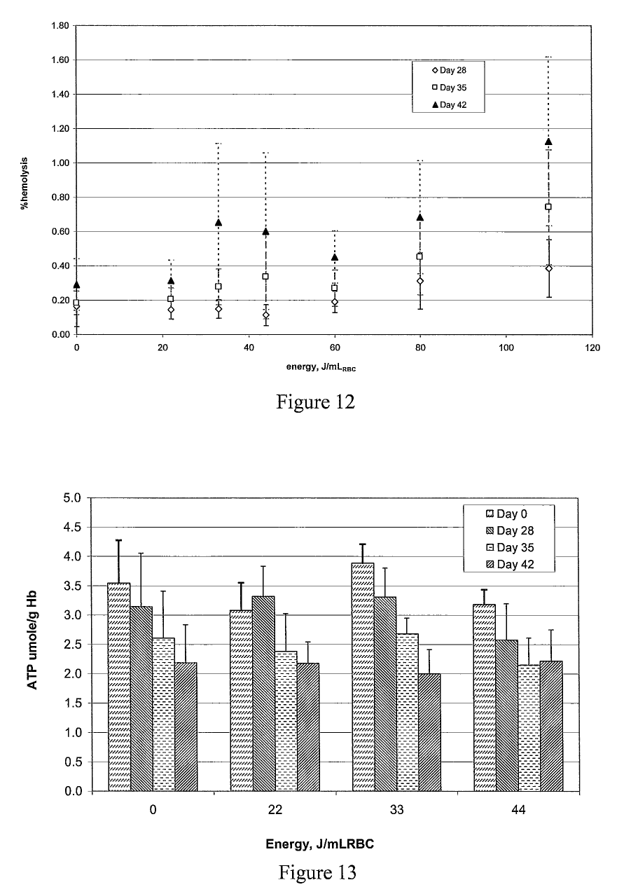

As shown in FIG. 12, increased energy levels decrease the total concentration of ATP in the cells.

### EXAMPLE 6

Osmotic fragility during storage of red blood cells separated from pathogen inactivated whole blood was measured. The normal red blood cell is a relatively impermeable biconcave disc which maintains osmotic equilibrium with the surrounding medium. As the surrounding medium becomes hypotonic, fluid will be taken into the cell to maintain stability. Eventually under very hypotonic conditions the cell will fill to capacity and rupture. Red blood cells with damaged membranes have a decreased capacity to expand, and will rupture in mildly hypotonic conditions that fail to lyse normal red cells. They thus exhibit increased osmotic fragility. Mean osmotic fragility (MOF) is a measure of red blood cell membrane fragility. The higher the MOF, the more fragile the red blood cells are. MOF is the concentration of NaCl where 50% of the red blood cells hemolyze.

As can be seen in FIG. 13, treated red blood cells separated from illuminated whole blood which was then stored for 42 days were on average slightly more fragile than untreated cells.

### EXAMPLE 7

Measurement of potassium concentration in stored red blood cells is another measure of red blood cell viability. Potassium leaks out of the red blood cells when the potassium pump in the red blood cell membrane is not working correctly. Damage to the potassium pump may occur during a pathogen inactivation procedure.

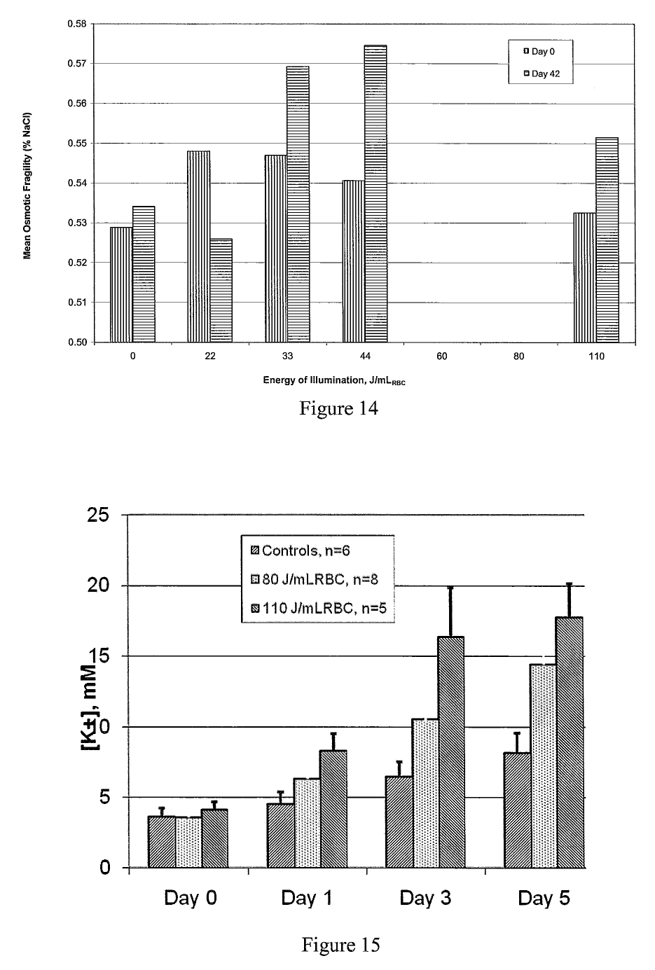

The potassium concentration of whole blood (not separated blood components) stored at room temperature over 5 days was measured. As shown in FIG. 14, the effects of energy on red blood cell integrity increase as the energy increases.

### EXAMPLE 8

Plasma was separated from pathogen inactivated whole blood and the quality of the plasma proteins was measured on day 0 and day 28.

As seen in FIGS. 15A and 15B, percent activity or concentration of plasma proteins appears to decrease in treated plasma at 110 J/mL_{RBC}, as compared to untreated plasma and treated plasma at lower energies. This occurs at both day 0 and day 28.

### EXAMPLE 9

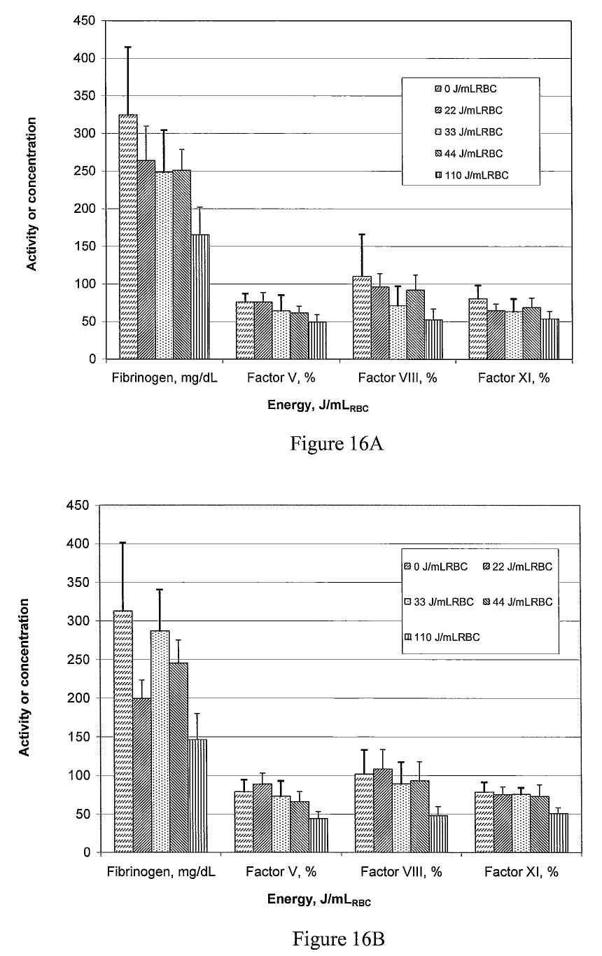

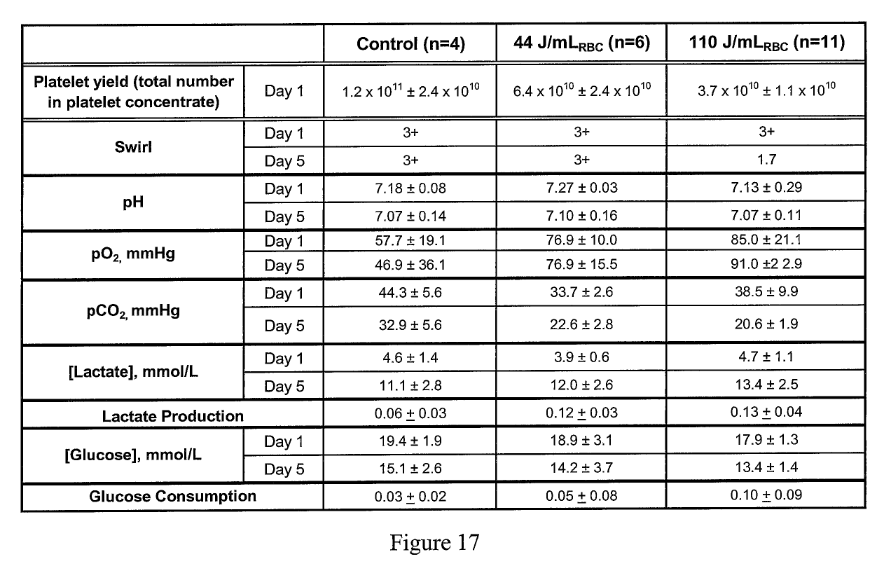

Platelets were separated from pathogen inactivated whole blood and markers of platelet quality were measured. The results are shown in FIG. 16. While pH remains the same over 5 days of storage for both treated and untreated cells, oxygen consumption appears to increase in treated platelets over 5 days of storage at higher energy levels, as does lactate production, while carbon dioxide production and glucose consumption appears to decrease at higher energy levels over 5 days of storage.

From the results above, blood components separated from pathogen inactivated whole blood appear to be viable even when illuminated at a variety of energy levels.

1. A method of pathogen inactivating whole blood comprising the steps of:
collecting whole blood from a donor into a bag;
illuminating the whole blood with light at a sufficient energy so that an alloxazine photosensitizer present in the whole blood may be photolyzed to inactivate any pathogens which may be present in the whole blood; and
storing the pathogen inactivated whole blood.

2. The method of claim 1 further comprising adding the alloxazine photosensitizer to the bag before the whole blood is collected.

3. The method of claim 1 further comprising adding the alloxazine photosensitizer to the bag after the whole blood is collected.

4. The method of claim 1 wherein the alloxazine photosensitizer is riboflavin.

5. The method of claim 1 wherein the energy sufficient to photolyze the alloxazine photosensitizer in the whole blood is between about 22-110 J/mL_{RBC}.

6. The method of claim 1 wherein the whole blood is illuminated in the collection bag.

7. The method of claim 1 wherein the whole blood is illuminated in a bag separate from the collection bag.

8. The method of claim 1 further comprising a step of separating the stored pathogen inactivated whole blood into separated blood components.

9. The method of claim 8 wherein the whole blood is separated into components in the collection bag.

10. The method of claim 8 wherein the whole blood is separated into components in a bag separate from the collection bag.

11. The method of claim 8 wherein the separated blood components further comprise red blood cells.

12. The method of claim 8 wherein the separated blood components further comprise platelets.

13. The method of claim 8 wherein the separated blood components further comprise plasma.

14. A method of collecting, pathogen inactivating and separating whole blood into at least one pathogen reduced blood component comprising the steps of:
collecting whole blood in a bag;
illuminating the whole blood and an alloxazine photosensitizer for a time sufficient to inactivate any pathogens which may be present in the whole blood to create pathogen inactivated whole blood;
separating the pathogen inactivated whole blood into at least one blood component and
expressing the at least one blood component.

15. The method of claim 14 wherein the alloxazine photosensitizer is riboflavin.

16. The method of claim 14 wherein the at least one blood component is red blood cells.

17. The method of claim 14 wherein the at least one blood component is platelets.

18. The method of claim 14 wherein the at least one blood component is plasma.

19. The method of claim 14 wherein the photosensitizer is added to the bag before the whole blood is collected.

20. The method of claim 14 wherein the photosensitizer is added to the bag after the whole blood is collected.

21. The method of claim 14 wherein the separation step is a centrifugation step.

22. The method of claim 14 wherein the collecting, illuminating, separating and expressing steps occur in the same bag.

23. The method of claim 14 wherein the collecting, illuminating, separating and expressing steps occur in separate bags.

24. The method of claim 16 wherein the red blood cells are not leukoreduced prior to administering them to a patient.

25. A method for separating pathogen inactivated whole blood into components comprising the steps of:
loading a bag containing pathogen inactivated whole blood onto a rotor;
spinning the rotor to separate the pathogen inactivated whole blood into at least a first component and a second component; and
squeezing the bag to transfer the first component into a first satellite bag.

26. The method of claim 25 further comprising the step of squeezing the bag to transfer the second component into a second satellite bag.

27. The method of claim 25 wherein the second component remains in the bag.

28. The method of claim 25 further comprising the step of spinning the rotor to further separate the pathogen inactivated whole blood into a third component.

29. The method of claim 25 wherein the step of squeezing the bag occurs on the rotor.

30. A pre-connected bag and solution set comprising:
a collection bag for collecting whole blood;
an illumination bag pre-connected via transfer tubing to the collection bag; and
a storage bag pre-connected via transfer tubing to the illumination bag.

31. The pre-connected bag and collection set of claim 28 further comprising a photosensitizer bag containing photosensitizer pre-connected via transfer tubing to the illumination bag.
